(19) United States
(12) Patent Application Publication
(10) Pub. No.: US 2026/0144458 A1
(43) Pub. Date: May 28, 2026

(54) SYSTEM AND A METHOD FOR PRODUCING INFORMATION INDICATIVE OF CARDIAC ABNORMALITY

(71) Applicant: Precordior Oy, Turku (FI)

(72) Inventors: Tero KOIVISTO, Turku (FI); Ismail ELNAGGAR, Turku (FI); Mikko SAVONTAUS, Turku (FI); Jouni PYKÄRI, Turku (FI); Tuija VASANKARI, Turku (FI)

(21) Appl. No.: 19/430,101

(22) Filed: Dec. 22, 2025

Related U.S. Application Data
(63) Continuation of application No. PCT/EP2025/067316, filed on Jun. 19, 2025.

(30) Foreign Application Priority Data
Jun. 25, 2024 (FI) 20247092

(51) Int. Cl. A61B 5/11; A61B 5/00

(52) U.S. Cl. CPC A61B 5/1126; A61B 5/7253; A61B 5/7264; A61B 5/7278; A61B 5/742; A61B 2562/0219

(57) ABSTRACT

Described are computer-implemented methods for determining a cardiac abnormality of an individual. The computer-implemented methods may include obtaining a signal indicative of cardiac motion and generated by a sensor. The computer-implemented methods may include identifying one or more features of the signal over a temporal length of the signal. The computer-implemented methods may include extracting one or more sample signal portions of the signal based at least in part on the one or more features. The computer-implemented methods may include applying an integral transform to the one or more sample signal portions. The computer-implemented methods may include determining a classification of a sample signal portion of the one or more sample signal portions based at least in part on a coefficient of the integral transform applied to the sample signal portion of the one or more sample signal portions.

### CROSS REFERENCE

This application is a continuation of International Patent Application No. PCT/EP2025/067316, filed Jun. 19, 2025, which claims the benefit of Finnish patent application No. FI20247092 filed Jun. 25, 2024, each of which are incorporated by reference herein in their entirety.

### FIELD

The invention relates generally to producing information indicative of cardiac abnormality, such as aortic stenosis “AS”, heart valve disease, heart failure, atrial fibrillation, and/or other heart conditions. More particularly, the invention relates to a system for producing information indicative of cardiac abnormality. Furthermore, the invention relates to a method and to a computer program for producing information indicative of cardiac abnormality.

### BACKGROUND

Abnormalities that may occur in the cardiovascular system, if not diagnosed and appropriately treated and/or remedied, may progressively decrease the ability of the cardiovascular system to maintain a blood flow that meets the needs of a body of an individual especially when the individual encounters physical stress. For example, aortic stenosis “AS” occurs when the aortic valve narrows, and blood cannot flow normally. The aortic stenosis is a common valvular heart disease affecting up to about 4% of elderly people. The aortic stenosis can lead to various complications, including heart failure and increased mortality if left untreated. In the latent period and early stage of disease, many patients are asymptomatic. Aortic murmur auscultation can still be detected, which prompts further investigation, cardiology referral and echocardiography. Observation is recommended for most asymptomatic patients when aortic stenosis is not yet severe. Symptomatic patients with severe aortic stenosis are usually referred for aortic valve replacement.

Currently, the main method for diagnosing aortic stenosis is echocardiography. The use of echocardiography is technically demanding and may require years of training. Furthermore, echocardiography machines are expensive. These factors limit its availability for screening large populations e.g. in an outpatient setting. Therefore, there exists a need for techniques for producing information indicative of cardiac abnormality, such as aortic stenosis, without expensive equipment and specialized operating personnel.

### SUMMARY

The following presents a simplified summary to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

In accordance with the invention, there is provided a system for producing information indicative of cardiac abnormality, including but not limited to aortic stenosis “AS”, heart valve disease, heart failure, and atrial fibrillation. The system comprises:
a signal interface for receiving a motion signal indicative of cardiac motion and generated by a motion sensor system, e.g. an accelerometer and/or a gyroscope, having a mechanical contact with a chest of an individual, and
a processing system coupled to the signal interface and configured to:
   extract, from the motion signal, samples each having a temporal length of one or more heart-beat periods so that a temporal phase of heart-beat related features, such as a closure of mitral and tricuspid valves “S₁” and a closure of aortic and pulmonary valves “S₂”, with respect to a beginning of the sample is same within each of the samples having the features,
   compute an integral transform of each of the samples to map each of the samples to basis-functions of the integral transform, and
   classify the samples to one of predefined categories, such as e.g. a healthy case, a cardiac abnormality case, and an inconclusive i.e. undefined case, based on coefficients resulting in the integral transform of each of the samples, set an indicator signal to express a result of the classification, and output the indicator signal.

The indicator signal can be transmitted to the individual and can include an instruction to seek treatment and/or advice from a healthcare provider. Alternatively or in addition, the indicator signal can be transmitted to a healthcare provider and can be an indication of the presence of a cardiac abnormality in the individual. The indicator signal may comprise cardiac measurement data, such as the motion signal, transmitted to a healthcare provider.

The above-mentioned classification can be performed by a machine learning classification algorithm, such as the Gradient Boosting Classifier “GBC”, that is trained with a training database comprising first data collected, with a motion sensor, e.g. an accelerometer and/or a gyroscope, from heathy persons and second data collected from persons having cardiac abnormality such as e.g. aortic stenosis. The Gradient Boosting Classifier is based on a functional gradient algorithm that repeatedly selects a function that leads in the direction of a weak hypothesis or negative gradient so that it can minimize a loss function. The Gradient boosting classifier combines several weak learning models to produce a powerful predicting model. It is also possible to use other classification algorithms for classifying the samples to correspond either a healthy case or a cardiac abnormality based on the coefficients resulting in the integral transform of each of the samples. Thus, the invention is not limited to any specific classification methods. The integral transform can be for example the continuous wavelet transform “CWT” where the basis-functions are differently time-shifted and time-stretched or -shrunk versions of a base-wavelet, or the Fourier transform where the basis-functions are sinusoidal functions, e.g. exponential functions having imaginary arguments, with different frequencies, or the Laplace transform where the basis-functions are exponential functions having complex-valued arguments. Thus, the invention is not limited to any specific integral transforms. The coefficients resulting in the integral transform are typically scalar products between the samples and the basis-functions of the integral transform. The classification can be performed by a classification system comprising a signal processing model, a signal matching model, and a classification model. The signal processing model can be configured to generate a temporal length value for each of one or more signal samples. The signal matching model can receive input from the signal processing model comprising the one or more signal samples. The signal matching model can predict at least one corresponding point on each of the signal samples. The signal matching model can map the one or more corresponding points of each of the signal samples to create a signal sample stack. The signal matching model can output data comprising the signal sample stack to the classification model. The classification model can generate a prediction of an appropriate classification category for the signal sample stack from one or more features of each signal sample. The classification model can output an indication of the classification category, which can be a cardiac abnormality case, a healthy case, or an inconclusive case.

In some embodiments, the signal matching model can generate feedback output comprising data indicating that one or more signal samples are nonconforming samples. The signal processing model can receive the feedback data from the classification model and adjust one or more temporal length values. The classification model can provide categorizing feedback output data to the signal matching model. The signal matching model can adjust mapping of the one or more signal samples based on the categorizing feedback output data

The system for producing the information indicative of cardiac abnormality may comprise a sensor system comprising one or more accelerometers for generating the above-mentioned motion signal indicative of cardiac motion and/or one or more gyroscopes for generating the above-mentioned motion signal indicative of cardiac motion. It is also possible that the signal interface is configured to receive the motion signal from an external apparatus comprising an appropriate sensor system, i.e. it is emphasized that the system does not necessarily comprise means for generating the motion signal indicative of the cardiac motion. The system can be for example a smartphone or another hand-held apparatus comprising an accelerometer and/or a gyroscope. A smartphone can be e.g. an Apple iPhone, an Android phone, a Google Pixel phone, Motorola phone, or another type of smartphone. The smartphone or the other hand-held apparatus can be placed on an individual's chest to generate the above-mentioned motion signal caused by the motion of the heart. The system can comprise, for example, a patch or wearable sensor able to contact the individual's chest when the individual is laying prone. The system can comprise a medical provider apparatus or other handheld medical apparatus.

In this document, the term “accelerometer” covers sensors of various kinds for measuring acceleration of a linear transverse motion. An accelerometer can be for example a microelectromechanical system “MEMS” based on the law of inertia. In this document, the term “gyroscope” covers sensors of various kinds for measuring angular rotations. A gyroscope can be for example a microelectromechanical system “MEMS” based on an effect of the Coriolis force acting on a back-and-forth turning object inside the MEMS gyroscope.

In accordance with the invention, there is also provided a method for producing information indicative of cardiac abnormality, such as aortic stenosis, heart valve disease, heart failure, and/or atrial fibrillation. The method comprises:
receiving a motion signal indicative of cardiac motion and generated by a motion sensor system having a mechanical contact with a chest of an individual,
extracting, from the motion signal, samples each having a temporal length of one or more heart-beat periods so that a temporal phase of heart-beat related features with respect to a beginning of the sample is same within each of the samples having the features,
computing an integral transform of each of the samples to map each of the samples to basis-functions of the integral transform, and
classifying the samples to one of predefined categories, such as e.g. a healthy case, a cardiac abnormality case, and an inconclusive case, based on coefficients resulting in the integral transform of each of the samples, setting an indicator signal to express a result of the classification, and outputting the indicator signal.

In accordance with the invention, there is also provided a computer program for producing information indicative of cardiac abnormality, such as aortic stenosis, heart valve disease, heart failure, and/or atrial fibrillation. The computer program comprises computer executable instructions for controlling a programmable processing system to:
receive a motion signal indicative of cardiac motion and generated by a motion sensor system having a mechanical contact with a chest of an individual,
extract, from the motion signal, samples each having a temporal length of one or more heart-beat periods so that a temporal phase of heart-beat related features with respect to a beginning of the sample is same within each of the samples having the features,
compute an integral transform of each of the samples to map each of the samples to basis-functions of the integral transform, and
classify the samples one of predefined categories, such as e.g. a healthy case, a cardiac abnormality case, and an inconclusive case, based on coefficients resulting in the integral transform of each of the samples, set an indicator signal to express a result of the classification, and output the indicator signal.

In some embodiments, the invention can comprise a system for generating an indication of a cardiac condition of an individual, the system comprising:
a signal interface configured to receive input comprising a motion signal indicative of cardiac motion generated by a motion sensor system having a mechanical contact with a chest of an individual, and
a processing system configured to:
   i) identify one or more characteristic heart-beat related features of each of samples of the motion signal over a temporal length of the motion signal,
   ii) apply an integral transform to each of the samples to map each of the samples to basis-functions of the integral transform,
   iii) classify the samples into one of categories based on coefficients resulting in the integral transform of each of the samples, and
   iv) output an indicator signal comprising a result of the classification of the samples.

In some embodiments, the processing system further configured to select a subset of the samples from the motion signal, wherein the subset of the samples have corresponding characteristic heart-beat related features.

In some embodiments, the motion sensor system can comprise an accelerometer, a gyroscope, or both. In some embodiments, the one or more characteristic heartbeat related features can comprise one or more of an amplitude feature, a frequency feature, a power feature, an intensity feature, an energy feature, or any combination thereof. In some embodiments, the categories can comprise a healthy case, a cardiac abnormality case, and an inconclusive case.

In some embodiments, the indicator signal can further comprise one or more of a notification of the predictive classification into a category, an indication to contact a healthcare professional based on the predictive classification, data output concerning the motion signal indicative of cardiac motion, or a display of the motion signal indicative of cardiac motion, or any combination thereof.

In some embodiments, the invention can comprise a method for generating an indication of a cardiac condition of an individual, the method comprising:
a signal interface configured to receive input comprising a motion signal indicative of cardiac motion generated by a motion sensor system having a mechanical contact with a chest of an individual,
identifying one or more characteristic heart-beat related features of each of samples of the motion signal over a temporal length of the motion signal,
applying an integral transform to each of the samples to map each of the samples to basis-functions of the integral transform,
classifying the samples into one of categories representing different cardiac conditions based on coefficients resulting in the integral transform of each of the samples
output an indicator signal comprising a result of the classification of the samples.

In some embodiments, the method further comprises selecting a subset of the samples from the motion signal, wherein the subset of the samples have corresponding characteristic heart-beat related features.

In some embodiments, receiving input by the motion sensor system can comprise receiving input from an accelerometer, a gyroscope, or both. In some embodiments, identifying the one or more characteristic features can further comprise identifying one or more of an amplitude feature, a frequency feature, a power feature, an intensity feature, an energy feature, or any combination thereof. In some embodiments, the categories can comprise a healthy case, a cardiac abnormality case, and an inconclusive case. In some embodiments, outputting the indicator signal can further comprise outputting an indication to contact a healthcare professional based on the classification, outputting data concerning the signal indicative of cardiac motion, or outputting a display of the motion signal indicative of cardiac motion, or any combination thereof.

In some embodiments, the invention can comprise a system for indicating a cardiac condition, the system comprising:
an input model configured to receive input comprising a motion signal from a motion sensor system having a mechanical contact with a chest of an individual,
a sample identification model configured to:
   a) identify one or more characteristic heart-beat related features of the motion signal received from the input model over a temporal length of the signal, and
   b) generate one or more samples of the motion signal by mapping the one or more characteristic features of the one or more samples received from the input model,
a classification model configured to generate a prediction of a classification of the one or more samples to one of categories based on integral transform coefficients of each of the one or more samples.
an output model configured to output an indication of the classification received from the classification model of the one or more samples.

In some embodiments, the input received from the motion sensor system can comprise input from an accelerometer, a gyroscope, or both. In some embodiments, the one or more characteristic heart-beat related features can comprise one or more of an amplitude feature, a frequency feature, a power feature, an intensity feature, an energy feature, or any combination thereof. In some embodiments, the categories can comprise a healthy case, a cardiac abnormality case, and an inconclusive case. In some embodiments, the signal can further comprise one or more of: an indication to contact a healthcare professional based on the classification, data output concerning the signal indicative of cardiac motion, or a display of the signal indicative of cardiac motion, or any combination thereof.

In some embodiments, the sample identification model is further configured to adjust the identification of the one or more characteristic features based on feedback data from the classification model. In some cases, the feedback data can comprise an indication that one or more characteristic features are nonconforming. In some cases, the feedback data can comprise an indication that one or more characteristic features are not relevant.

In some embodiments the sample identification model can be further configured to generate one or more modified samples based on feedback data from the classification model. In some cases, the feedback data can comprise data indicating that one or more samples are nonconforming. In some cases, the feedback data can comprise data indicating that one or more samples are noise samples.

In some embodiments the one or more modified samples can be generated from modification of the mapping of the one or more characteristic features of the one or more samples. In some cases, the mapping can be modified by excluding one or more samples from the mapping. In some embodiments, the mapping can be modified by using a different characteristic feature for the mapping.

In some embodiments, the classification model can be further configured to generate one or more modified classifications based on feedback data from the sample identification model. In some embodiments, the output model is further configured to generate an updated indication of the modified classification.

In accordance with the invention, there is also provided a computer program product. The computer program product comprises a non-volatile computer readable medium, e.g. a compact disc “CD”, encoded with a computer program according to the invention, a flash drive encoded with a computer program according to the invention, or a digital download of a computer program according to the invention.

A computer readable medium, such as computer-executable code, may take many forms, including but not limited to, a tangible storage medium, a carrier wave medium, or physical transmission medium. Non-volatile storage media may include, for example, optical or magnetic disks, such as any of the storage apparatuses in any one or more computers or the like, such as may be used to implement the databases. Volatile storage media include dynamic memory, such as main memory of such a computer platform. Tangible transmission media include coaxial cables, copper wire, and fiber optics, including the wires that comprise a bus within a computer system. Carrier-wave transmission media may take the form of electric or electromagnetic signals, or acoustic or light waves such as those generated during radio frequency “RF” and infrared “IR” data communications. Common forms of computer-readable media therefore include for example: a floppy disk, a flexible disk, a hard disk, a magnetic tape, any other magnetic medium, a compact disc read only memory “CD-ROM”, digital video disc “DVD” or DVD-ROM, any other optical medium, punch cards paper tape, any other physical storage medium with patterns of holes, a random access memory “RAM”, a read only memory “ROM”, a programmable read only memory “PROM”, and an erasable programmable read only memory “EPROM”, a flash-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Exemplifying and non-limiting embodiments are described in accompanied dependent claims.

Exemplifying and non-limiting embodiments both as to constructions and to methods of operation, together with additional objects and advantages thereof, will be best understood from the following description of specific exemplifying embodiments when read in conjunction with the accompanying drawings.

The verbs “to comprise” and “to include” are used in this document as open limitations that neither exclude nor require the existence of also un-recited features.

The features recited in the accompanied dependent claims are mutually freely combinable unless otherwise explicitly stated.

Furthermore, it is to be understood that the use of “a” or “an”, i.e. a singular form, throughout this document does not exclude a plurality. As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount, in some cases near the stated amount by 10%, 5%, or 1%, including increments therein, and in some cases, in reference to a percentage, refers to an amount that is greater or less the stated percentage by 10%, 5%, or 1%, including increments therein.

As used herein, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C”, “one or more of A, B, or C” and “A, B, and/or C” means A alone, B alone, C alone, A and B together, A and C together, B and C together, or A, B and C together.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

### BRIEF DESCRIPTION OF FIGURES

Exemplifying and non-limiting embodiments and their advantages are explained in greater detail below with reference to the accompanying drawings, in which:

FIG. 1a shows a schematic illustration of a system according to an exemplifying and non-limiting embodiment for producing information indicative of cardiac,

FIG. 1b shows exemplifying signals generated in the system shown in FIG. 1a,

FIG. 1c shows a functional block diagram of the system shown in FIG. 1a, and

FIG. 2 is a flow chart of a method according to an exemplifying and non-limiting embodiment for producing information indicative of cardiac abnormality.

### DESCRIPTION OF EXEMPLIFYING AND NON-LIMITING EMBODIMENTS

The specific examples provided in the description below should not be construed as limiting the scope of the invention. Lists and groups of examples provided in the description are not exhaustive unless otherwise explicitly stated.

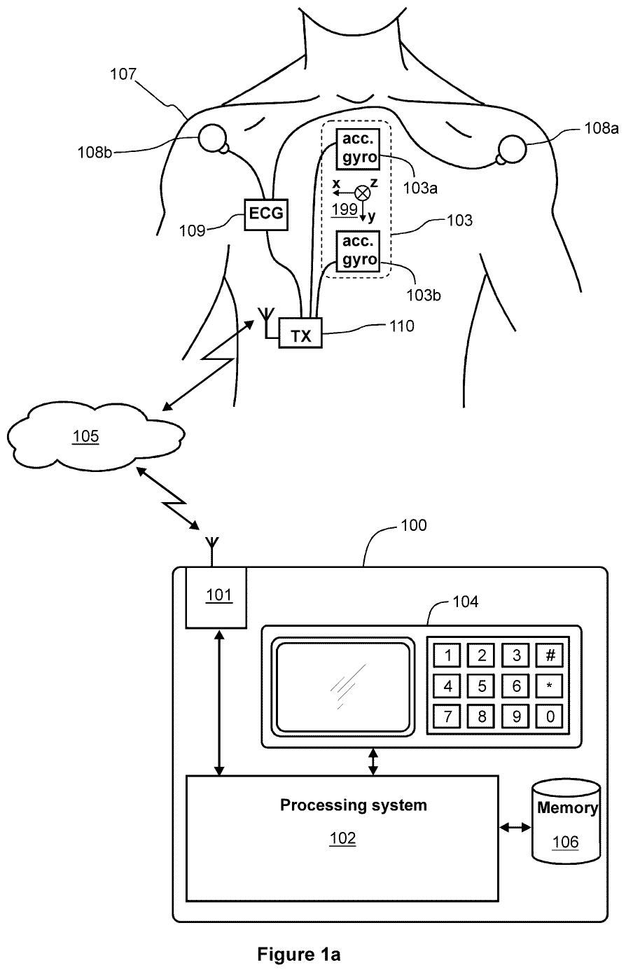

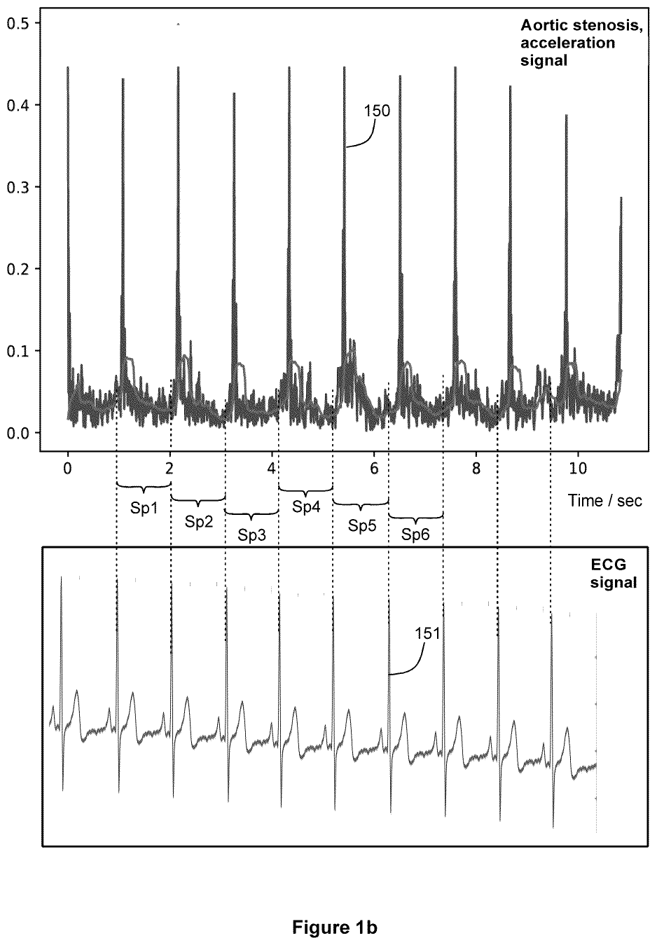

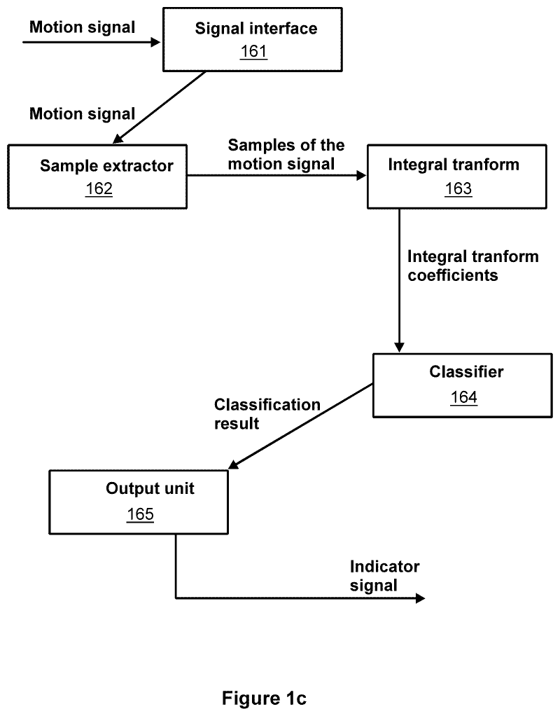

FIG. 1a shows a schematic illustration of a system 100 according to an exemplifying and non-limiting embodiment for producing information indicative of cardiac abnormality, such as aortic stenosis, heart valve disease, heart failure, and/or atrial fibrillation. The system 100 comprises a signal interface 101 for receiving a motion signal indicative of cardiac motion and generated by a motion sensor system 103 having a mechanical contact with a chest of an individual 107. The system 100 comprises a processing system 102 coupled to the signal interface 101. The processing system 102 is configured to:
extract, from the motion signal, samples each having a temporal length of one or more heart-beat periods so that a temporal phase of heart-beat related features, such as a closure of mitral and tricuspid valves “S₁” and a closure of aortic and pulmonary valves “S₂”, with respect to a beginning of the sample is same within each of the samples having the features,
compute an integral transform of each of the samples to map each of the samples to basis-functions of the integral transform, and
classify the samples to one of predefined categories, such as e.g. a healthy case, a cardiac abnormality case, and an inconclusive case, based on coefficients resulting in the integral transform of each of the samples and set an indicator signal to express a result of the classification.

The above-mentioned sensor system 103 comprises at least one accelerometer and/or at least one gyroscope. It is also possible that the sensor system 103 comprises for example one or more inertial measurement units “IMU” each comprising both an accelerometer and a gyroscope each of which can be for example a microelectromechanical system “MEMS”. The temporal duration of the motion signal can be, for example but not necessarily, between tens of seconds and many days. In some cases, the temporal duration of the signal can be about 10 seconds, about 20 seconds, about 30 seconds, about 40 seconds, about 50 seconds, about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, about 24 hours, about 2 days, about 3 days, about 4 days, about 5 days, about 6 days, about 7 days, or more than about 7 days.

The above-mentioned indicator signal that is output by the system 100 can be for example a message shown on a display screen of a user-interface 104. The indicator signal may comprise an instruction to seek treatment and/or advice from a healthcare provider. The system 100 can be configured to transfer the indicator signal to a healthcare provider. Furthermore, cardiac measurement data such as e.g. a recorded waveform of the motion signal indicative of cardiac motion can be transmitted to the healthcare provider.

In the exemplifying case illustrated in FIG. 1a, the sensor system 103 is connected to the signal interface 101 via a transmitter 110 and one or more data transfer links each of which can be for example a radio link or a corded link. The data transfer from the transmitter 110 to the signal interface 101 may take place either directly or via a data transfer network 105 such as e.g. a telecommunication network. In the exemplifying case illustrated in FIG. 1a, the transmitter 110 is a radio transmitter. It is also possible that the system comprising the processing system 102 is integrated with the sensor system, i.e. the system 100 contains the sensor system. In this exemplifying case, the system is configured to generate the motion signal and the signal interface can be a simple wiring from the sensor system to the processing system. A system comprising an integrated sensor system can be for example a smartphone or another hand-held apparatus which can be placed on the chest of an individual during a measurement phase. The smartphone can be e.g. an Apple iPhone, an Android phone, a Google Pixel phone, Motorola phone, or another type of smartphone. The hand-held apparatus can be for example a patch or wearable sensor able to contact the individual's chest when the individual is laying prone. The hand-held apparatus can be a medical provider apparatus or another handheld medical apparatus.

A system according to an exemplifying and non-limiting embodiment is configured to record the motion signal indicative of cardiac motion. The recorded motion signal can be generated within a time window having a fixed temporal start-point and a fixed temporal end-point, or within a sliding time window having a fixed temporal length and moving along with elapsing time. The system may comprise an internal memory 106 for recording the signal and/or the system may comprise a data port for connecting to an external memory. The system may comprise a wireless transceiver for transmitting and receiving data wirelessly to and from an external memory.

In a system according to an exemplifying and non-limiting embodiment, the processing system 102 is configured to compute a continuous wavelet transform “CWT” of each of the samples. The basis-functions of CWT are differently time-shifted and time-scaled versions of a base-wavelet i.e. a mother-wavelet, and the CWT coefficients are scalar products between the samples and the basis-functions of the CWT transform. The time-scaling of the base-wavelet is expressed by a scale parameter, whereas the time-shifting of the base-wavelet is expressed by a translational value. Thus, each CWT coefficient is related to a specific scale parameter value and to a specific translational value.

In a system according to an exemplifying and non-limiting embodiment, the processing system 102 is configured to compute a Fourier transform of each of the samples. The basis-functions of the Fourier transform are exponential functions having imaginary arguments with different frequencies, and the Fourier coefficients are scalar products between the samples and the basis-functions of the Fourier transform.

In a system according to an exemplifying and non-limiting embodiment, the processing system 102 is configured to implement a Gradient Boosting Classifier “GBC” to classify the samples to correspond either a healthy case or a cardiac abnormality case, such as e.g. aortic stenosis, or to be inconclusive. The Gradient Boosting Classifier “GBC” is a machine learning classification algorithm that is trained with a training database comprising first data collected from heathy persons and second data collected from persons having cardiac abnormality, e.g. aortic stenosis.

In a system according to an exemplifying and non-limiting embodiment, the processing system 102 is configured to apply a classification algorithm, e.g. the above-mentioned GBC, directly to the coefficients resulting in the integral transforms of the samples, i.e. without preprocessing the coefficients, in order to classify the samples to correspond either a healthy case or a cardiac abnormality case, such as e.g. aortic stenosis, or to be inconclusive. In a system according to another exemplifying and non-limiting embodiment, the processing system 102 is configured to preprocess the coefficients resulting in the integral transforms and apply the classification algorithm to results of the preprocessing. An exemplifying preprocessing in a case where the integral transform is the continuous wavelet transform “CWT” is presented below.

The application of the CWT on a sample of the motion signal results in a matrix of coefficients representing the information of the sample in the time-frequency domain. This matrix captures the intensity and distribution of frequencies in the sample across time so that a first dimension of the matrix corresponds to different scale parameter values, i.e. different frequency bands, of the CWT and the second dimension of the matrix corresponds to different translational values of the CWT. By extracting specific features from these coefficients, insights into the underlying hemodynamics can be obtained and potential abnormalities can be detected.

The following three exemplifying features 1)-3) can be extracted for each scale parameter value of the CWT and for each of the samples corresponding to different time-windows across the motion signal:
1) Max Amplitude: This feature identifies the maximum amplitude of the CWT coefficients for each scale parameter value. It can help capture of dominant frequency components within each sample.
2) Mean Value: Computing the mean value of the CWT coefficients for each scale parameter value gives a measure of the average power or intensity of that frequency component over time.
3) Total Energy: This is the sum of the squared CWT coefficients at each scale parameter value. It represents the total energy or power in the motion signal for a specific scale parameter.

Computation of the above-mentioned three features 1)-3) is illustrated below with the following example array that could be a set of coefficients obtained with the CWT for a given sample so that i) the CWT has a given scale parameter value and ii) the translational value of the CWT is varied to have ten different values, i.e. the array is a single row of the above-mentioned matrix of coefficients:
[1, 2, 3, 4, 5, 6, 7, 8, 9, 10].

In this exemplifying case, the Max Amplitude is the largest absolute value in the array=Max{abs(1, 2, 3, 4, 5, 6, 7, 8, 9, 10)}=10.

In this exemplifying case, the Mean Value is the average of all the numbers in the array=Mean{1, 2, 3, 4, 5, 6, 7, 8, 9, 10}=5.5.

In this exemplifying case, the Total Energy is the sum of the squares of the numbers in the array=1²+2²+3²+4²+5²+6²+7²+8²+9²+10²=385.

Thus, for the above-presented exemplifying array, the computed three features 1)-3) are:
Max Amplitude: 10
Mean Value: 5.5
Total Energy: 385.

These features can be computed for every value of the scale parameter of the CWT, and for every sample of the motion signal. Then, the computed features can be fed to a classification algorithm, e.g. the Gradient Boosting Classifier.

Same preprocessing, such as the above-presented example, is advantageously applied both on training data for training the classification algorithm and on data based on which a detection of possible cardiac abnormality, e.g. aortic stenosis, is carried out.

In a system according to an exemplifying and non-limiting embodiment, the processing system 102 is configured to compute the integral transforms of the samples without preprocessing the samples. In a system according to another exemplifying and non-limiting embodiment, the processing system 102 is configured to preprocess the samples prior to computing the integral transforms of the samples. The preprocessing may comprise for example filtering the samples with a filter that suppresses frequency components which are outside a normal frequency band of the samples, and which thereby represent noise.

In a system according to an exemplifying and non-limiting embodiment, the processing system 102 is configured to carry out dynamic time warping “DTW” to measure similarity between the samples and to reject those of the samples which differ most from a majority of the samples. Thus, the samples which are most badly corrupted with noise can be discarded from the further processing, i.e. the integral transform and the classification. It is however also possible to use other methods to weed out samples corrupted with noise.

In a system according to an exemplifying and non-limiting embodiment, the signal interface 101 is configured to receive an electrocardiogram “ECG” signal and the processing system 102 is configured to find, from the electrocardiogram signal, R-peaks and to determine the temporal length of the samples based on a temporal difference between successive ones of the R-peaks. It is also possible to detect the heart-beat rate from the motion signal based on e.g. aortic opening “AO” and/or aortic closure “AC” peaks, and/or other heart-beat related features in the motion signal.

In the exemplifying case illustrated in FIG. 1a, there is single lead ECG system comprising electrocardiogram electrodes 108a and 108b and a differential amplifier 109 configured to form the ECG signal. The transmitter 110 is configured to transfer the ECG signal to the signal interface 101 in addition to the motion signal. It is also possible that the ECG system is a part of the system 100 so that the system 100 comprises the ECG electrodes 108a and 108b, the differential amplifier 109, and wires connecting the ECG electrodes to the differential amplifier.

In the exemplifying embodiment illustrated in FIG. 1a, the motion signal comprises a first component and a second component. The first component of the motion signal is generated by a first motion sensor 103a that is a part of the motion sensor system 103 and has a mechanical contact with a first place on the chest of the individual 107. The second component of the motion signal is generated by a second motion sensor 103b that is a part of the motion sensor system 103 and has a mechanical contact with a second place on the chest of the individual 107. In the exemplifying situation shown in FIG. 1a, the first motion sensor 103a is on an upper part of the sternum of the individual 107 and the second motion sensor 103b is on a lower part of the sternum of the individual 107. The first motion sensor 103a may comprise an accelerometer and/or a gyroscope. Correspondingly, the second motion sensor 103b may comprise an accelerometer and/or a gyroscope. The measurement from two different places on the chest of the individual 107 improves the reliability of detection of cardiac abnormality e.g. aortic stenosis. In an exemplifying case where each of the first and second motion sensors 103a and 103b comprises both an accelerometer and a gyroscope, the motion signal has four signal components. Each of these signal components may have three subcomponents if the respective accelerometer or gyroscope is a three-axis accelerometer or gyroscope. The integral transform can be computed for each subcomponent separately, or the sub-components can be combined with e.g. the following formula and thereafter the integral transform can be computed for the result r obtained with the formula:

$$ri = {q}_{x i}^{2} + {q}_{y i}^{2} + {q}_{z i}^{2} , \qquad ( 1 )$$

where i is an index increasing with time, qₓᵢ is an i^{th} value of an x-component of the cardiac motion, e.g. acceleration in the x-direction of a coordinate system 199 or rotation around a geometric axis parallel with the x-axis of the coordinate system 199, qₓᵢ is an i^{th} value of a y-component of the cardiac motion in the coordinate system 199, and q_{zi} is an i^{th} value of a z-component of the cardiac motion in the coordinate system 199.

FIG. 1b shows an exemplifying waveform of a motion signal 150 generated by a three-axis accelerometer and using the above-presented formula 1 so that qₓᵢ, q_{yi}, and q_{zi}, are accelerations at a time moment i in the x-, y-, and z-directions of the coordinate system 199, respectively. Furthermore, FIG. 1b shows an electrocardiogram “ECG” signal 151. In this exemplifying case, the processing system 102 is configured to find, from the electrocardiogram signal, R-peaks and the samples of the motion signal 151 are portions of the motion signals 150 between successive ones of the R-peaks. In FIG. 1b, exemplifying samples are denoted as Sp1, Sp2, Sp3, Sp4, Sp5, and Sp6.

FIG. 1c shows a functional block diagram of the system shown in FIG. 1a. A functional block 161 is a signal interface for receiving the motion signal indicative of cardiac motion, a functional block 162 is a sample extractor for extracting, from the motion signal, the samples, a functional block 163 is an integral transform computer for computing the integral transform of each of the samples, a functional block 163 is a classifier for classifying the samples to one of the predefined categories based on the coefficients resulting in the integral transforms of the samples, and the functional block 163 is an output unit for setting an indicator signal to express a result of the classification and outputting the indicator signal. The functional blocks 161-165 can represent software modules and/or corresponding parts of a hardware processor.

The processing system 102, and thereby the functional blocks shown in FIG. 1c, can be implemented for example with one or more processor circuits, each of which can be a programmable processor circuit provided with appropriate software, a dedicated hardware processor such as, for example, an application specific integrated circuit “ASIC”, or a configurable hardware processor such as, for example, a field programmable gate array “FPGA”. The memory 106 can be implemented for example with one or more memory circuits, each of which can be e.g. a random-access memory “RAM” apparatus.

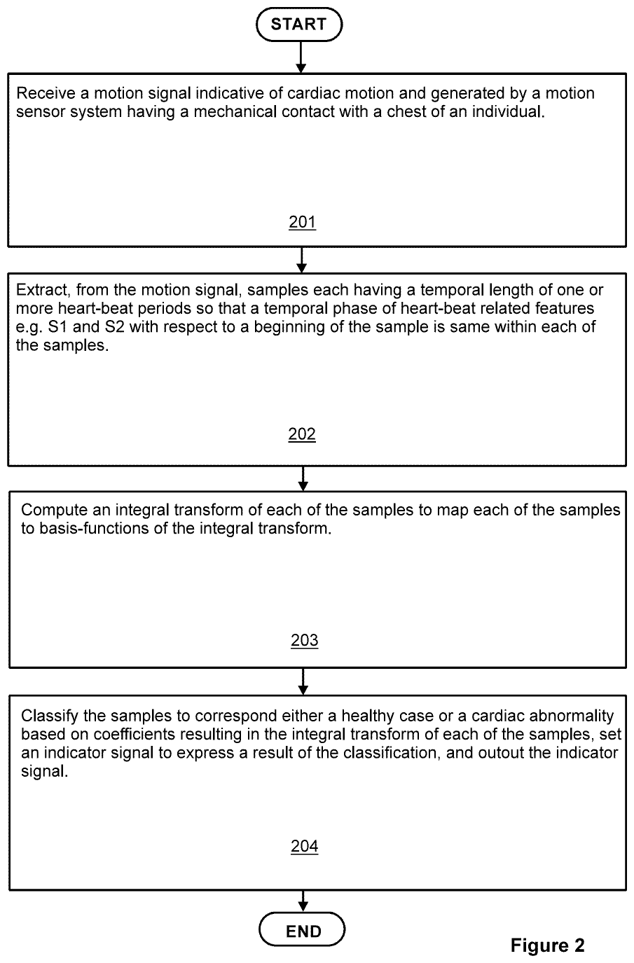

FIG. 2 shows a flow chart of a method according to an exemplifying and non-limiting embodiment for producing information indicative of cardiac abnormality, such as aortic stenosis, heart valve disease, heart failure, and/or atrial fibrillation. The method comprises the following actions:
action 201: receiving a motion signal indicative of cardiac motion and generated by a motion sensor system having a mechanical contact with a chest of an individual,
action 202: extracting, from the motion signal, samples each having a temporal length of one or more heart-beat periods so that a temporal phase of heart-beat related features with respect to a beginning of the sample is same within each of the samples having the features,
action 203: computing an integral transform of each of the samples to map each of the samples to basis-functions of the integral transform, and
action 204: classifying the samples to one of predefined categories, such as e.g. a healthy case, a cardiac abnormality case, and an inconclusive case, based on coefficients resulting in the integral transform of each of the samples and setting an indicator signal to express a result of the classification.

A method according to an exemplifying and non-limiting embodiment comprises the generation of the above-mentioned motion signal by the motion sensor system having the mechanical contact with the chest of the individual. A method according to another exemplifying and non-limiting embodiment comprises reading this motion signal from a memory, in which case the motion signal has been generated earlier and recorded in the memory. A method according to an exemplifying and non-limiting embodiment comprises receiving the motion signal from an external data transfer system. Therefore, the generation of the above-mentioned motion signal, i.e. a measuring step, is not an essential and necessary step of methods according to embodiments of the invention.

A method according to an exemplifying and non-limiting embodiment comprises computing the integral transforms of the samples without preprocessing the samples.

A method according to another exemplifying and non-limiting embodiment comprises preprocessing the samples prior to computing the integral transforms of the samples. The preprocessing may comprise for example filtering the samples.

In a method according to an exemplifying and non-limiting embodiment, the integral transform of each of the samples is a continuous wavelet transform “CWT” of the sample.

In a method according to an exemplifying and non-limiting embodiment, the integral transform of each of the samples is a Fourier transform of the sample.

A method according to an exemplifying and non-limiting embodiment comprises applying a classification algorithm, e.g. the GBC, directly to the coefficients resulting in the integral transforms of the samples, i.e. without preprocessing the coefficients, in order to classify the samples to correspond to a healthy case or a cardiac abnormality case, such as e.g. aortic stenosis, or to be inconclusive.

A method according to another exemplifying and non-limiting embodiment comprises preprocessing the coefficients resulting in the integral transforms and applying the classification algorithm to the results of the preprocessing.

A method according to an exemplifying and non-limiting embodiment comprises carrying out dynamic time warping “DTW” to measure similarity between the samples and rejecting those of the samples which differ most from the majority of the samples.

In a method according to an exemplifying and non-limiting embodiment, the motion signal comprises a first component that is generated by a first motion sensor being a part of the motion sensor system and having a mechanical contact with a first place, e.g. on an upper part of a sternum, on the chest of the individual, and a second component that is generated by a second motion sensor being a part of the motion sensor system and having a mechanical contact with a second place, e.g. on a lower part of the sternum, on the chest of the individual.

In a method according to an exemplifying and non-limiting embodiment, at least a part of the motion signal is generated by an accelerometer having the mechanical contact with the chest of the individual.

In a method according to an exemplifying and non-limiting embodiment, at least a part of the motion signal is generated by a gyroscope having the mechanical contact with the chest of the individual.

A method according to an exemplifying and non-limiting embodiment comprises receiving an electrocardiogram “ECG” signal and finding, from the electrocardiogram signal, R-peaks and to determine the temporal length of the samples based on a temporal difference between successive ones of the R-peaks.

In a method according to an exemplifying and non-limiting embodiment, the cardiac abnormality is aortic stenosis.

A computer program according to an exemplifying and non-limiting embodiment comprises computer executable instructions for controlling a programmable processing system to carry out a method according to any of the above-presented exemplifying and non-limiting embodiments.

A computer program according to an exemplifying and non-limiting embodiment comprises software modules for producing information indicative of cardiac abnormality, such as aortic stenosis, heart valve disease, heart failure, atrial fibrillation, and/or other cardiac abnormality. The software modules comprise computer executable instructions for controlling a programmable processing system to:
receive a motion signal indicative of cardiac motion and generated by a motion sensor system having a mechanical contact with a chest of an individual,
extract, from the motion signal, samples each having a temporal length of one or more heart-beat periods so that a temporal phase of heart-beat related features with respect to a beginning of the sample is same within each of the samples having the features,
compute an integral transform of each of the samples to map each of the samples to basis-functions of the integral transform, and
classify the samples to one of predefined categories, such as e.g. a healthy case, a cardiac abnormality case, and an inconclusive case, based on coefficients resulting in the integral transform of each of the samples.

The software modules can be e.g. subroutines or functions implemented with a suitable programming language and with a compiler suitable for the programming language and for the programmable processing system. It is worth noting that a source code corresponding to a suitable programming language represents the computer executable software modules because the source code contains the information needed for controlling the programmable processing system to carry out the above-presented actions and compiling changes only the format of the information. Furthermore, it is also possible that the programmable processing system is provided with an interpreter so that a source code implemented with a suitable programming language does not need to be compiled prior to running.

A computer program product according to an exemplifying and non-limiting embodiment comprises a computer readable medium, e.g. a compact disc “CD”, encoded with a computer program according to an embodiment of the invention, a flash drive encoded with a computer program according to the invention, or a digital download of a computer program according to the invention.

A computer readable medium according to an exemplifying and non-limiting embodiment is encoded with a computer program according to an embodiment of invention.

A computer readable medium, such as computer-executable code, may take many forms, including but not limited to, a tangible storage medium, a carrier wave medium or physical transmission medium. Non-volatile storage media include, for example, optical or magnetic disks, such as any of the storage apparatuses in any one or more computers or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency “RF” and infrared “IR” data communications. Common forms of computer-readable media therefore include for example: a floppy disk, a flexible disk, hard disk, magnetic tape, any other magnetic medium, a compact disc read only memory “CD-ROM”, digital video disc “DVD” or DVD-ROM, any other optical medium, punch cards paper tape, any other physical storage medium with patterns of holes, a random access memory “RAM”, a read only memory “ROM”, a programmable read only memory “PROM” and an erasable programmable read only memory “EPROM”, a flash-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

A signal according to an exemplifying and non-limiting embodiment is encoded to carry information defining a computer program according to an embodiment of invention.

In some embodiments, a system for indicating a cardiac condition can comprise an input model configured to receive input comprising a motion signal from a motion sensor system having a mechanical contact with a chest of an individual. In some embodiments, the motion signal can be read from a memory, in which case the motion signal had been measured earlier and recorded in the memory. In some embodiments, the motion signal can be received from an external data transfer system. Therefore, the measuring is not an essential and necessary step according to embodiments of the invention. In some embodiments, the input can be received from an accelerometer, a gyroscope, or both.

In some embodiments, the system can further comprise a sample identification model configured to:
identify one or more characteristic heart-beat related features of the motion signal received from the input model over a temporal length of the signal, and
generate one or more samples of each of the motion signals by mapping the one or more characteristic features of the one or more samples received from the input model.

In some embodiments, the one or more characteristic features comprise one or more of an amplitude feature, a frequency feature, a power feature, an intensity feature, an energy feature, or any combination thereof. In some embodiments, the characteristic features can be heart-beat related features, such as a closure of mitral and tricuspid valves “S1” and a closure of aortic and pulmonary valves “S2”. In some embodiments, the features can comprise Max Amplitude: This feature identifies the maximum amplitude of the CWT coefficients for each scale parameter value. It can help capture of dominant frequency components within each sample. In some embodiments, the features can comprise Mean Value: Computing the mean value of the CWT coefficients for each scale parameter value gives a measure of the average power or intensity of that frequency component over time. In some embodiments, the features can comprise Total Energy: This is the sum of the squared CWT coefficients at each scale parameter value. It represents the total energy or power in the motion signal for a specific scale parameter.

In some embodiments, the system can further comprise a classification model configured to generate a prediction of a classification of the samples to one of categories based on integral transform coefficients of the samples.

In some embodiments, the classification model can apply integral transforms to the samples without preprocessing the samples. In some embodiments, the classification model can preprocess the samples and then apply the integral transforms to the preprocessed samples. The preprocessing may comprise for example filtering the samples. In some embodiments, the integral transform of each of the samples can be a continuous wavelet transform “CWT” of the sample. In some embodiments, the integral transform of each of the samples can be a Fourier transform of the sample.

In some embodiments, the classification model can apply a classification algorithm directly to the coefficients resulting in the integral transforms of the samples. In some embodiments, preprocessing the coefficients can be carried out and the classification algorithm can be applied to results of the preprocessing. In some embodiments, a dynamic time warping “DTW” function can be applied to measure similarity between the samples and rejecting those of the samples which differ most from the majority of the samples.

In some embodiments, the categories can comprise a healthy case, a cardiac abnormality case, and an inconclusive case.

In some embodiments, the system can further comprise an output model configured to output an indicator signal expressing the classification result received from the classification model. In some embodiments, the indicator signal can further comprise one or more of: an indication to contact a healthcare professional based on the classification result, data concerning the motion signal indicative of cardiac motion, or a display of the motion signal indicative of cardiac motion, or any combination thereof.

In some embodiments, the signal can be output as a visual notification, audio notification, or tactile notification, or any combination thereof.

In some embodiments, the sample identification model can be further configured to adjust the identification of the one or more characteristic features based on feedback data from the predictive classification model. In some embodiments, the feedback data can comprise nonconforming data. In some embodiments, the feedback data can comprise categorizing the samples into the inconclusive case, the healthy case, or the cardiac abnormality case.

In some embodiments, the sample identification model can be further configured to predictively generate one or more modified samples based on feedback data from the predictive classification model. In some embodiments, the feedback data can comprise nonconforming data. In some embodiments, the feedback data can comprise categorizing the samples into the inconclusive case, the healthy case, or the cardiac abnormality case.

In some embodiments, the one or more modified samples can be generated from modification of the mapping of the one or more characteristic features of the one or more samples. In some embodiments, one or more samples can be excluded from the mapping. In some embodiments, one or more samples can be duplicated in the mapping.

In some embodiments, the classification model can be further configured to generate one or more modified classifications based on feedback data from the sample identification model. In some embodiments, the feedback data can further comprise one or more nonconforming samples or data points.

In some embodiments, the output model can be further configured to generate an updated indication of the modified classification. In some embodiments, the updated indication can comprise an indication of a classification of the samples to one of: a cardiac abnormality case, a healthy case, and an inconclusive case. In some embodiments, the indication can be an error message. In some embodiments, the indication can be a request to generate an additional motion signal.

In some embodiments, a system for generating an indication of a cardiac condition of an individual comprises:
a signal interface configured to receive input comprising a signal indicative of cardiac motion generated by a motion sensor system having a mechanical contact with a chest of an individual; and
a processor configured to:
   i) identify one or more characteristic features of each of the motion signals over a temporal length of the signal,
   ii) apply an integral transform to each of the samples to map each of the samples to basis-functions of the integral transform,
   iii) classify the samples into one of predefined categories based on coefficients of the integral transform of each of the samples, and
   iv) output a signal comprising a result of the classification of the samples.

In some embodiments, the processor is further configured to select a subset of samples from the motion signals, wherein the subset of the samples have corresponding characteristic features.

In some embodiments, the motion sensor system comprises an accelerometer, a gyroscope, or both.

In some embodiments, the one or more characteristic features comprise one or more of an amplitude feature, a frequency feature, a power feature, an intensity feature, an energy feature, or any combination thereof.

In some embodiments, the one or more categories comprise a healthy case, a cardiac abnormality case, and an inconclusive case.

In some embodiments, the signal further comprises one or more of: an indication to contact a healthcare professional based on the classification, data output concerning the signal indicative of cardiac motion, or a display of the signal indicative of cardiac motion, or any combination thereof.

In some embodiments, a method of generating an indication of a cardiac condition of an individual comprises:
receiving input comprising a signal indicative of cardiac motion generated by a motion sensor system having a mechanical contact with a chest of an individual,
identifying one or more characteristic features of each of the motion signals over a temporal length of the signal,
applying an integral transform to each of the samples to map each of the samples to basis-functions of the integral transform,
classifying the samples into one of predefined categories based on coefficients of the integral transform of each of the samples, and
outputting a signal comprising a result of the classification of the samples.

In some embodiments, the method further comprises selecting a subset of the samples from the motion signals, wherein the subset of the samples have corresponding characteristic features.

In some embodiments, the receiving the input from the motion sensor system comprises receiving input from an accelerometer, a gyroscope, or both.

In some embodiments, identifying the one or more characteristic features further comprises identifying one or more of an amplitude feature, a frequency feature, a power feature, an intensity feature, an energy feature, or any combination thereof.

In some embodiments, the categories comprise a healthy case, a cardiac abnormality case, and an inconclusive case.

In some embodiments, outputting the signal further comprises one or more of: outputting an indication to contact a healthcare professional based on the predictive classification, outputting data concerning the signal indicative of cardiac motion, or outputting a display of the signal indicative of cardiac motion, or any combination thereof.

In some embodiments, a system for indicating a cardiac condition comprises:
i) an input model configured to receive input comprising a motion signal from a motion sensor system having a mechanical contact with a chest of an individual,
ii) a sample identification model configured to:
   a) identify one or more characteristic features of each of the motion signals received from the input model over a temporal length of the signal, and
   b) generate one or more samples of each of the motion signals by mapping the one or more characteristic features of the one or more samples received from the input model,
iii) a classification model configured to classify the samples to one of predefined categories based on coefficients of the integral transform of each of the one or more samples, and
iv) an output model configured to output a result of the classification received from the predictive classification model.

In some embodiments, the input received from the motion sensor system comprises input from an accelerometer, a gyroscope, or both.

In some embodiments, the one or more characteristic features comprise one or more of an amplitude feature, a frequency feature, a power feature, an intensity feature, an energy feature, or any combination thereof.

In some embodiments, the categories comprise a healthy case, a cardiac abnormality case, and an inconclusive case.

In some embodiments, the signal further comprises one or more of: an indication to contact a healthcare professional based on the predictive classification, data output concerning the signal indicative of cardiac motion, or a display of the signal indicative of cardiac motion, or any combination thereof.

In some embodiments, the sample identification model is further configured to adjust the identification of the one or more characteristic features based on feedback data from the predictive classification model In some embodiments, wherein the sample identification model is further configured to predictively generate one or more modified samples based on feedback data from the predictive classification model.

In some embodiments, the one or more modified samples are generated from modification of the mapping of the one or more characteristic features of the one or more samples.

In some embodiments, the predictive classification model is further configured to generate one or more modified classifications based on feedback data from the sample identification model.

In some embodiments, wherein the output model is further configured to generate an updated indication of the modified classification.

### Example Case

In the example case, the Gradient Boosting Classifier “GBC” was trained using a training dataset collected from 106 individuals. 57 of these individuals were diagnosed with aortic stenosis “AS”, and 49 of the individuals have a history of cardiac disease excluding valve heart diseases and heart failure. The motion signals related to the training dataset were signals generated by an accelerometer on chests of the individuals. The integral transform used in the training phase was the continuous wavelet transform “CWT”, and the features Max Amplitude, Mean Value, and Total Energy were computed as presented earlier in this document and these features were fed to the Gradient Boosting Classifier.

A test dataset was collected from additional 106 age and sex matched individuals. Of these 106 individuals, 7 individuals were excluded because of issues during data collection. These issues consisted of poor signal quality, e.g. a poor signal-to-noise ratio “SNR”, during recording or missing information including the true status of the individuals when the recording was taken. This left 99 individuals. The motion signals related to the test dataset were signals generated by an accelerometer on chests of the individuals.

Table 1 shows results achieved for the test dataset with a method according to an embodiment where the integral transform is the continuous wavelet transform “CWT”, the above-mentioned features Max Amplitude, Mean Value, and Total Energy are computed and fed to the Gradient Boosting Classifier “GBC” trained with the above-mentioned training dataset. The predictions by the method were checked by a cardiologist. In Table 1, a True Positive is a person with aortic stenosis “AS” being correctly diagnosed with AS and a True Negative is a person without AS being correctly diagnosed without AS:

TABLE 1

| | Predicted Positive | Predicted Negative |
|---|---|---|
| True Positive | 46 | 4 |
| True Negative | 6 | 43 |

### Remarks

The specific examples provided in the description given above should not be construed as limiting the scope of the invention. Lists and groups of examples provided in the description given above are not exhaustive unless otherwise explicitly stated.

What is claimed is:

1. A computer-implemented method for determining a cardiac abnormality of an individual comprising:
(a) obtaining a signal indicative of cardiac motion, wherein the signal is generated by a sensor;
(b) identifying one or more features of the signal over a temporal length of the signal;
(c) extracting one or more sample signal portions of the signal based at least in part on the one or more features;
(d) applying an integral transform to the one or more sample signal portions;
(e) determining a classification of a sample signal portion of the one or more sample signal portions based at least in part on a coefficient of the integral transform applied to the sample signal portion of the one or more sample signal portions; and
(f) generating an indication of the cardiac abnormality based at least in part on the determined classification of (e).

2. The computer-implemented method of claim 1, wherein the determining of the classification of (e) further comprises selecting a subset of the one or more sample signal portions, wherein the subset of the one or more sample signal portions have the same coefficient of the integral transform.

3. The computer-implemented method of claim 1, wherein the sensor comprises an accelerometer, a gyroscope, or both.

4. The computer-implemented method of claim 1, wherein the identifying of the one or more features of the signal further comprises identifying one or more of: an amplitude feature, a frequency feature, a power feature, an intensity feature, an energy feature, or any combination thereof.

5. The computer-implemented method of claim 1, wherein determining the classification of (e) further comprising determining one or more categories of the classification, wherein the one or more categories comprise: a healthy case, a cardiac abnormality case, or an inconclusive case.

6. The computer-implemented method of claim 1, wherein generating the indication further comprises generating one or more of: an indication to contact a healthcare professional based on the predictive classification, data concerning the signal indicative of cardiac motion, or a display of the signal indicative of cardiac motion, or any combination thereof.

7. A computer-implemented method for determining a cardiac abnormality, the method comprising:
(a) obtaining a signal associated with cardiac motion of an individual, wherein the signal is measured by one or more sensors;
(b) extracting a plurality of signal sample portions from the motion signal;
(c) generating a signal sample stack comprising a subset of the signal sample portions having the same temporal length;
(d) determining the cardiac abnormality based at least in part on comparing one or more features of the subset of the signal sample portions of the signal sample stack to one or more corresponding reference features; and
(e) generating an indication of the cardiac abnormality.

8. The computer-implemented method of claim 7, wherein the one or more sensors comprise an accelerometer, or a gyroscope, or both.

9. The computer-implemented method of claim 7, further comprising, prior to generating the signal sample stack, determining a temporal length of at least a portion of the plurality of signal sample portions of the signal.

10. The computer-implemented method of claim 9, wherein generating the signal sample stack further comprises selecting the subset of the signal sample portions based at least in part on the temporal length and one or more corresponding points of the selected subset of the signal sample portions.

11. The computer-implemented method of claim 10, wherein generating the signal sample stack further comprises determining a signal sample portion of the plurality of signal sample portions is a nonconforming signal sample portion.

12. The computer-implemented method of claim 11, further comprising determining the nonconforming signal sample portion based at least in part determining one or more points of the nonconforming signal sample portion do not correspond to the selected subset of the signal sample portions.

13. The computer-implemented method of claim 12, wherein generating the signal sample stack further comprises removing the nonconforming signal sample portion from the signal sample stack.

14. The computer-implemented method of claim 12, wherein generating the signal sample stack further comprises modifying the temporal length of the nonconforming signal sample portion, thereby generating a modified signal sample portion.

15. The computer-implemented method of claim 14, wherein generating the signal sample stack further comprises determining one or more points of the modified signal sample portion correspond to one or more points of the selected subset of the signal sample portions.

16. The computer-implemented method of claim 14, wherein generating the signal sample stack further comprises adding the modified signal sample portion to the signal sample stack.

17. The computer-implemented method of claim 7, wherein the temporal length comprises one or more heart-beat periods.

18. The computer-implemented method of claim 7, wherein the one or more features of the subset of the signal sample portions of the signal sample stack comprise one or more of: an amplitude feature, a frequency feature, a power feature, an intensity feature, an energy feature, or any combination thereof.

19. The computer-implemented method of claim 7, wherein the cardiac abnormality comprises aortic stenosis (AS), heart valve disease, heart failure, or atrial fibrillation, or any combination thereof.

20. A system for determining a cardiac abnormality of an individual comprising:
(a) a signal interface configured to obtain a signal indicative of cardiac motion measured by a sensor; and
(b) one or more processors configured to:
(i) identify one or more features of the signal over a temporal length of the signal;
(ii) extract one or more sample signal portions of the signal based at least in part on the one or more features;
(iii) apply an integral transform to the one or more sample signal portions;
(iv) determine a classification of a sample signal portion of the one or more sample signal portions based at least in part on a coefficient of the integral transform applied to the sample signal portion of the one or more sample signal portions; and
(v) generate an indication of the cardiac abnormality based at least in part on the classification of (iv).

21. A system for determining a cardiac abnormality, the system comprising:
(a) a signal interface configured to obtain a signal indicative of cardiac motion measured by a sensor; and
(b) one or more processors configured to:
(i) extract a plurality of signal sample portions from the motion signal,
(ii) generate a signal sample stack comprising a subset of the signal sample portions having the same temporal length,
(iii) determine the cardiac abnormality based at least in part on comparing one or more features of the subset of the signal sample portions of the signal sample stack to one or more corresponding reference features, and
(iv) generate an indication of the cardiac abnormality.
